(19) United States
(12) Patent Application Publication
(10) Pub. No.: US 2024/0264166 A1
(43) Pub. Date: Aug. 8, 2024

(54) Unique non-invasive process for detecting and identifying cancers and diseases within humans and animals

(71) Applicant: Calvin T. Kutzer, CARSON CITY, NV (US)

(72) Inventor: Calvin T. Kutzer, CARSON CITY, NV (US)

(21) Appl. No.: 17/803,956

(22) Filed: Feb. 6, 2023

(51) Int. Cl. G01N 33/68; G01N 21/65

(52) U.S. Cl. CPC G01N 33/6851; G01N 21/65

(57) ABSTRACT

My new process utilizing “Raman Spectroscopy”, in which a white laser light is passed through air and moisture molecules shed by humans and animals to detect and identify cancers and diseases will greatly benefit the field of medical diagnostics.

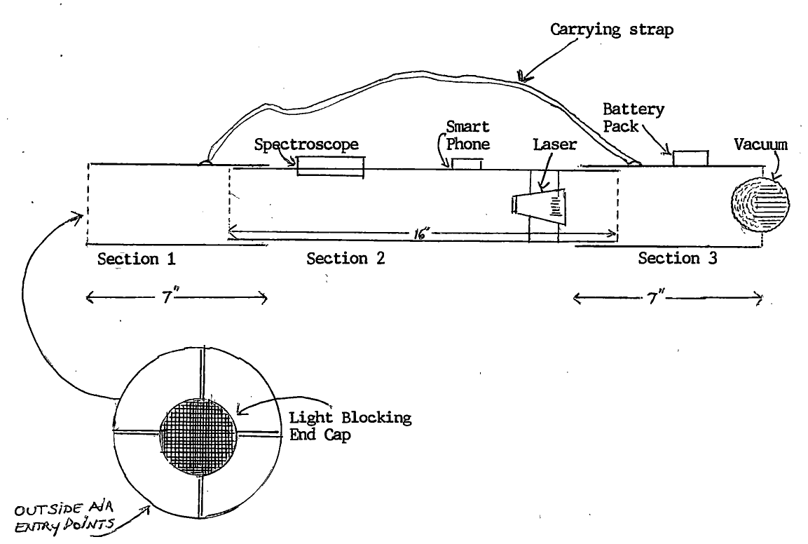

Molecular signatures are the cohesive groups of repeating wavelength patterns of the molecules shed from a known or unknown source as revealed on a spectroscope.

Comparing by computer, the spectroscopic group patterns of similar and repeating wavelengths of molecularly scattered white laser light from molecules shed by people and animals, with those wavelengths previously recorded on computer, of the molecular patterns of various cancers and diseases, a matching pattern or molecular signature, between a cancer or disease with the molecular pattern from molecules shed by a subject person, will then reveal either a positive correlation with cancer or disease, or, a negative correlation.

This unique process will save thousands of lives yearly.

### BACKGROUND OF THE INVENTION

Molecules are the building blocks of all materials, substances, and organic matter.

All materials, substances, and organic matter lose molecules naturally, which are shed, in a miniscule, yet steady stream, continually.

When a dog finds a lost person by smelling that person's trail, it is due to the dog's ultra-keen sense of smell, which can pick up the scent of the shed molecules of that lost person.

In other words the dog is detecting through smell, the unique molecular signature of that person.

Some dogs have a sense of smell so sensitive that there have been occurrences in which they have detected cancers and diseases in their owners through their sense of smell.

When a dog detects a cancer or disease in its owner by smell, it indicates that molecules making up that cancer or disease have escaped from or been shed by the dog's owner's body, through sweat, breath, pores in the skin, and other ways, which has allowed the dog to now detect those molecules.

Which, once again, by smell, the dog is detecting the actual molecular scent or signature of those cancers and diseases themselves.

### BRIEF SUMMARY OF THE INVENTION

Another more versatile and transportable means of detecting such cancerous and diseased molecules emanating from people, is through “Spectroscopy”, using the “Raman Effect”.

Not to be confused with “Mass Spectroscopy” which is an entirely different form of spectroscopy which uses a magnet to separate a beam of ions by mass, nor should it be confused with Infrared Raman Spectroscopy used in forensics, Fourier-Transform Infrared Spectroscopy, Flame Spectrometry, or, with CAM Ion-Mobility Spectrometry.

When a white laser light passes through transparent gas, such as air, or even transparent solids or liquids, such as blood plasma and serum, the molecules will scatter the lasers light in a pattern consistent with the material from which they originated.

### Molecular Signature Detector (M.S. Detector)

A M.S. Detector will then disclose not only a spectral line associated with that lasers light, but also, will display other lines of varying wave lengths associated with the molecules present.

This “Raman Spectrum” can then be used to detect different cancers and diseases non-invasively by a spectrographic analysis of laser light scattered by the molecules shed from that person, and, then comparing those molecular signatures with the pre-programmed molecular signature groupings of different cancers and diseases preprogrammed on a smartphone and at a central computer database.

This method can then be used to identify a multitude of different types of cancers, diseases, one of a kind individual human molecular signatures for identification purposes including other organic materials spectrographically, yet, of only those things which have already had their molecular signatures ascertained and then recorded onto a smartphone, and, onto a computer data base, to which a sample may be compared.

### BRIEF DESCRIPTION OF THE DRAWING

A simple M.S. Detector can be assembled using different configurations.

A goal is to construct a miniaturized high-tech version of a M.S. Detector which ultimately, any health care professional could carry with them, as they do now with smartphones, giving medical professionals the unique ability to determine, with a high degree of accuracy any cancer or disease which could be present in a subject patient.

Today's sophisticated technologies will certainly make a small miniaturized M.S. Detector with a miniature laser and Spectroscope incorporated within, very doable.

Operating components need to include (1) A miniature white light laser (non-burning variety) (2) A miniature spectroscope with a direct hardwired link to (3) An exterior mounted smart phone, with which will through a to be developed APP be used to both analyzed data on-site, and, transmit and stream (4) The Spectroscopes read out data directly back to the preprogrammed central computer for additional comparative analysis. And (5), a battery pack, and (6) A small miniaturized battery powered suction vacuum.

The battery pack can be affixed to the outside of the upper housing section, same as the smartphone.

This simplifies battery/electric needs of the components, eases battery replacement, and provides a visual of the various wires by using color coded wires leading to the components.

It also provides quick ready access to the smartphone.

NOTE: Though I describe below how to build a basic M.S. Detector configuration with modern technology, it will be easy to build a more efficient and more technologically advanced miniaturized M.S. Detector more suitable for today's needs.

### A Basic Molecular Signature Detector's Components, Housing, and Assembly

Built in sections so that it can be taken apart and put back together by hand, quickly, and easily, in order to facilitate component access for maintenance and upgrades.

Section 1, the lower “Sniffer” air intake section. Constructed of PVC pipe or any other similar material. This section needs to contain light blocking material across the bottom central portion of the inside diameter of the pipe, a removable snap-on end cap across the air-intake in such a way that it won't interfere with air intake yet will still prevent the white laser light from shining outside the bottom of the air intake opening.

Section 2, The middle insert. Constructed of the same material as section 1. The middle section insert will have a diameter of pipe slightly narrower to allow a snug insert fit into sections 1 and 3, into which it will be firmly slid.

The section 2 middle section insert will contain the miniature white light laser (non burning variety), and the special miniature spectroscope, which will continuously scan the lasers scattered light coming from the molecules vacuumed into the housing.

The light spectrum from the spectroscope is then fed by hardwire into an exterior mounted smartphone with gigabyte data storage of preprogrammed molecular signatures of cancers and diseases, which, also contains a special “APP” for further transmission or streaming of that spectroscopes data to a distant central computer for additional comparative analysis.

When spectra wavelengths on-site are matched with preprogrammed cancers and diseases through the smartphones comparison of the two, a signal initiated by the special APP is then sent through the smartphones input jacks to earbuds or headphones which will then signal an alert to health care professionals.

Similar to a metal detector's signal.

Section 3, The upper section of the housing. This section contains the important battery pack, which will provide electricity to all components.

The upper open end of the section 3 will have inserted into it the small battery powered vacuum, which is essential for pulling outside air up into the bottom of the unit, and then, up and through the white light laser shining directly in front sensors in the spectroscope, which will then analyze the molecular wavelength of the organic material, from the subject or patient over which the M.S. Detector is passed.

### DETAILED DESCRIPTION OF THE INVENTION

Using a simply built form of M.S. Detector, or in a sterile laboratory or hospital, molecules of various cancers and diseases can be quickly detected and shown to be present, and, then, each of those unique molecular arrangements will reveal the specific type of cancer or disease, which will then help determine its general location(s) in or on the body.

And, yes, this method will work quite well in Veterinary Medicine too. A M.S. Detector with attached smart phone, and too, a separately located central computer, both having the molecular signature groupings of different cancers and diseases recorded onto them both, will be programmed to ignore the molecules of unmatched and unrelated compounds, substances, and organic materials, with only the unique molecular signatures of known cancers and diseases being recognized, which match those present in the air, solid, or liquid being analyzed, especially molecules flowing from breath and skin pores.

This method can be roughly compared to the way fingerprints on-file are matched to unknown fingerprints left at a crime scene, yet, in this case, “Molecular Fingerprints” so-to-speak, from known cancers and diseases, recorded onto a central computer are compared to molecules vacuumed up by health care professionals from different patients, using a M.S. Detector.

With assistance from health care professionals, hospitals, and labs, a variety of cancerous tissues and fluids, and, diseased tissues and fluids, can be obtained, and then, under sterile controlled conditions, the unique molecular signature of virtually any cancer and any disease can then be ascertained.

This can be done by subjecting the evaporated moisture and organic gasses, or clear fluids of those known cancers and diseases, individually, in a controlled sterile environment: to eliminate contamination from outside molecules; to laser light to determine each ones unique molecular “Raman Spectrum” signature, through spectroscopy.

Then, those recorded molecular signatures will be added to others on a central data base, and, onto a special APP on smartphones being used in the field for such purposes.

It's likely that by using only a single sample in order to establish a true and accurate molecular signature “Standard” of any given cancer or disease, will result in other unrelated molecules inadvertently included as being a part of the actual molecular signature of those things.

Such molecules would be considered contaminating crossover molecules.

Crossover molecules would be associated with unrelated molecules from the molecular signature of the subject patient, from which the cancer or disease was obtained.

This would lead to the very real probability of a misreading and misinterpretation of spectra results.

Therefore, it is absolutely vitally important, that for each specific cancer and disease subjected to the “Raman Effect”, in order to obtain an accurate record of its molecular signature, that, at least 30 separate samples of each specific disease type be taken from 30 separate individual patients or deceased patients having had the exact same cancer or disease.

This would help to identify any contaminating crossover molecules from a sampled patient, and delete it from being inadvertently incorporated into the final recorded molecular signature.

Thus, one needs to compare each 30 (or 100) preliminary molecular signatures from each identical cancer and disease with all the others being sampled.

Take the same computer identified similar and repeating spectra wavelengths found in all 30 samples coming from each specific cancer and each specific disease, record them.

And then delete the non-similar and non-repeating wavelengths. For the non-similar and non-repeating wavelengths would logically be only from the crossover molecules.

It would then be a simple comparative matching study, by computer, to accurately identify the true molecular signature or molecular “Scent” of each specific cancer and each specific disease.

One could then conclude that if the same identical matching wavelengths are found in all 30 samples, from 30 different sampled subjects, which had been identified as all having originated from identical specific cancers and identical specific diseases, then those would be the true molecular signatures of any given cancer or disease.

Sterile samples of solid cancers, tumors, and diseases would be cut opened and then placed into a sterile glass container in a laboratory, then gently heated to cause slight evaporation, which would then allow a white laser light (non-burning type), to shine through an aperture directly into the glass container, and pass through the evaporated moisture and associated gas emanated from the sample.

A spectroscope directly outside the container would then record the molecular wavelength spectrum or signature of that evaporated organic gas.

Clear fluid samples of cancers and diseases would then be similarly processed, except they could either have the heated evaporated gasses and moisture shown through by a laser directly through the transparent fluid itself, to then have its molecular signature or spectrum, which is displayed on the spectroscope recorded onto a smartphone and a distant central computer database for later separate comparative molecular analysis.

With the data obtained bypassing a molecular signature detection over a person's body, and, “Vacuuming Up” air from that person coming directly from their exhaled breath, and, emanating from their skin and pores.

Then, comparing the molecules within such vacuumed up air to preprogrammed recorded molecular signatures of known cancers and diseases, prerecorded on both the gigabyte memory of the smartphone, and, in the central computers petabyte database, could then proceed in order to look for a molecular match.

Cancers and diseases are of different molecular types, species, and structure, depending upon where within a body (human or animal) such cancer or disease is located.

Therefore, not only will the “Raman Effect” detect the presence of a cancer or disease, but too, it will also identify the precise type of cancer or precise type of disease, based on the unique molecular grouping of each.

Pancreatic cancer has a different molecular grouping than does breast cancer, or lung cancer, or, brain cancer, or, one cancer, or, colon cancer, or other types of cancers, and, the same applies to diseases, which also have very distinctively different arrangements, or groupings, of molecules, depending on the disease type.

So, by recording, and then preprogramming the unique similar and repeating molecular groupings and patterns of all known cancers and diseases into a database, with the molecular signature of each, the detection of a preprogrammed cancer or disease will not only reveal its presence, but also its probable location, by also identifying and then matching the type, species, and structure of the specific type of cancer and specific type of disease, through its unique molecular arrangement and grouping. This will be of great help to medical professionals.

An identical yet more refined process using the “Raman Effect” can be used to detect cancerous and diseased molecular signatures within a person's transparent blood plasma/serum, to search for beginning cancers and diseases in an early stage of development.

This is done by taking a triple centrifuged sample of a person's nearly clear yellowish blood plasma/serum, placing it into a small sterile glass container with flat uncurved sides, (curved sides will distort the laser light and the molecules scattered light, which will ruin the analysis) then placing that small glass container in front of the spectroscope, and then subjecting it to the white laser light, within a controlled sterile laboratory setting. If any molecules, even a small number of any cancer or disease is inconclusive.

The spectroscope will then scan it, at which point, the diverse molecular spectrum wavelength groupings of all the molecules present in the plasma will then be transferred to the spectroscopes hardwired on-board smartphone, located on the outside of a M.S. Detector configuration which will then analyze all the molecular groupings present through its own gigabyte memory, and, through a specially developed “APP”, will also live stream the spectroscopes spectrum data readouts directly into the central preprogrammed petabyte computer, for backup analysis comparison.

### Using Molecular Signatures to Identify Individual People

All human beings consist of a unique combination and individualized assortment of molecules in one of a kind quantities and patterns.

Molecules from people are lost through sweat, breath exhalations, spit, blood/plasma, sluffing off of skin, scraping off the body, skin pores, evaporation, and through clothing, hats, shoes, and other emanations from the body in any number of ways.

Those lost molecules have the molecular signature of the person from whom they originated.

It's such a unique molecular signatures that hound dogs detect when put on the trail of game or missing persons.

And, it's the same unique molecular signature or molecular “Scent” found in humans, that can be used in a similar way as hound dogs do, to identify a person.

Therefore, similar and repeating wavelengths as described elsewhere in this specification can also be used to detect the very unique molecular signature, or scents, of people, which have been input into a database just as fingerprints and DNA are, and then used as an additional form of identification, for computer access, credit card security, investigations, and even for in-court evidence, and hearings of all types in which positive identification is important.

Just as tracking dogs can “Sniff Out” and follow the “Scent” of lost people, and single out the “Scent” of a particular individual person by their unique individualized molecular “Odor” or “Scent”, and, even identify a perpetrator who has left a crime scene, so too, may the “Raman Effect” be used, which can do those things and much much more.

This can be accomplished when using the Raman Effect on the person in a controlled laboratory setting when he or she is physically present, to identify their molecular signature, and then recording it on a Petabyte central computer data base.

The Raman Effect can also be used to identify the molecular signature of human hormones onto a M.S. Detector's smartphone “APP”, and, into the central computer.

The smartphones preprogrammed “APP”, and the central computer, also preprogrammed with all molecular signature data, will then allow them to be used to determine whether a male or female left a particular molecular signature behind, in a room, in a car, or, even handled an item.

The approximate age of a person can also be ascertained by analyzing the molecular pattern of their molecular signature and comparing that molecular pattern to ones found in various age groups of humans.

For, as a person ages, so do the molecular groupings within each person's body, thus, a fairly accurate age group can be determined by using the differences in molecular groupings in different age groups, and then comparing the subject's with those,

I claim:

1. The processes, applications, and uses of my new process and method of applying “Raman Spectroscopy” as I describe and explain within my enclosed “Specification”, “Drawing”, and “Abstract of the Disclosure” which I have incorporated hereby by reference.
